(19) United States
(12) Patent Application Publication
(10) Pub. No.: US 2006/0074464 A1
(43) Pub. Date: Apr. 6, 2006

(54) System and method for creating a customizable report of data from an implantable medical device

(76) Inventors: Steven J. Subera, St. Paul, MN (US); Bohdan O. Washchuk, Minneapolis, MN (US); Paul A. Blowers, Marine on St. Croix, MN (US); Seema Padmanabhan, Maple Grove, MN (US)

Correspondence Address: MEDTRONIC, INC., 710 MEDTRONIC PARK, MINNEAPOLIS, MN, 55432-9924, US

(21) Appl. No.: 10/973,101

(22) Filed: Oct. 25, 2004

Related U.S. Application Data
(60) Provisional application No. 60/589,251, filed on Jul. 20, 2004.

(51) Int. Cl. A61N 1/08

(52) U.S. Cl. 607/60

(57) ABSTRACT

Data is retrieved from an implantable medical device (IMD) for presentation by interrogating the IMD to access the data. Medical report parameters are established by user selection, and a medical report is assembled that includes only the accessed data that corresponds to the selected medical report parameters.

### CROSS-REFERENCE TO RELATED APPLICATION(S)

This application claims the benefit of U.S. Provisional Application No. 60/589,251 filed Jul. 20, 2004 for “System and Method for Creating a Variable Term Heart Failure Management Report” by S. Subera, B. Washchuk, P. Blowers and S. Padmanabhan.

### INCORPORATION BY REFERENCE

The aforementioned U.S. Provisional Application No. 60/589,251 is hereby incorporated by reference in its entirety.

### BACKGROUND OF THE INVENTION

The present invention relates to a medical reporting system for viewing data from an implantable medical device, and more particularly to a system for creating a medical report of implantable device data that is customizable by a user.

An important aspect of modern health care is the need to monitor the vital signs and other medical events and data associated with a patient, particularly those who employ an implantable medical device (IMD) of some kind in order to treat an illness or medical condition. Such monitoring has traditionally been performed by having a patient visit a hospital or clinic, so that a programmer or a similar device can interrogate the IMD to gather and display the information that the IMD has stored.

Recent developments in monitoring technology have made it possible for a patient to upload data from an IMD to a remote location via a communication network such as the worldwide web, using a telephone connection or a similar type of connection to transmit the information from the IMD to the remote location. One commercially available system for this type of communication is the CareLink® network provided by Medtronic, Inc. of Minneapolis, Minn. The remote monitoring provided by such systems allow a patient with an IMD to reduce the number and frequency of visits to a hospital or clinic, by periodically uploading data for review by a physician or other medical personnel to determine whether further follow up analysis is necessary. This capability gives patients significantly greater freedom in their lifestyle, and has brought a higher quality of life to many patients.

The data uploaded from an IMD to a remote monitoring location is typically available for display as a number of predetermined reports. For example, a monitoring physician can view a short-term, real-time intracardiac electrogram (EGM), or historical reports such as trended cardiac information (including, for example, atrial or ventricular tachyarrhythmias, average ventricular rate, and heart rate variability), episode reports showing the occurrence of particular types of medical events over time, or others. These reports, whether provided as the result of an in-office visit or as uploaded data from a remote location, are typically pre-rendered static images that have been compiled from the data provided by the IMD, and are typically shown over a fairly long time period, such as 14 months. This information allows the monitoring physician to analyze the need for further follow-up care for the patient, based on their historical medical trends and performance of their IMD.

There are some types of illnesses and conditions in which it would be useful for a monitoring physician to have more options available for viewing data provided by an IMD. For example, heart failure patients typically are scheduled for more frequent follow-up visits with a physician, and observing day-to-day changes in the patient's condition may be useful in determining what care is necessary. Furthermore, the time period for which the physician would like to view data and trends, as well as the types of data the physician would like to view together, may vary significantly from patient to patient, depending on their particular condition and history. It would be useful for a physician to have more individual control over the compilations and views of medical data, to allow the physician to make the best possible judgment in evaluating and advising the patient.

### BRIEF SUMMARY OF THE INVENTION

The present invention is a system and method for retrieving and presenting data from an implantable medical device (IMD). The IMD is interrogated to access the data. Medical report parameters are established by user selection, and a medical report is assembled that includes only the accessed data that corresponds to the selected medical report parameters. The user-defined medical report parameters include data types, a desired time period for the medical report, and viewing options for the medical report.

### BRIEF DESCRIPTION OF THE DRAWINGS

FIG. 1 is a diagram illustrating a communication system for the transmission of patient data to a clinician's station.

FIG. 2 is a Heart Failure Management Report rendered over a 14 month time period.

FIG. 3 is a Heart Failure Management Report that has 90 days of data, displayed on the 14 month time scale of a conventional report.

FIG. 4 is a flow diagram illustrating the process by which a user may create a custom report according to an embodiment of the present invention.

FIG. 5 is a custom Heart Failure Management Report created according to the process illustrated in FIG. 4.

### DETAILED DESCRIPTION

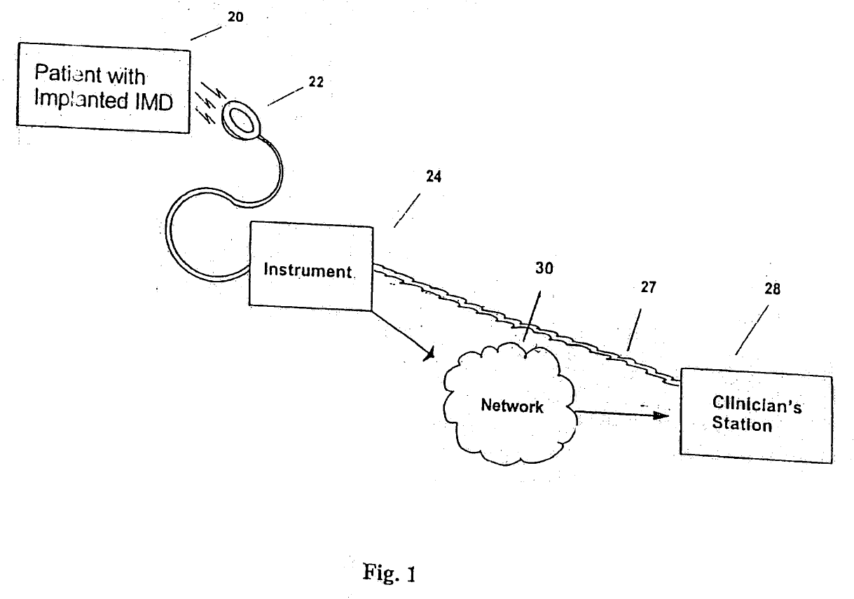

FIG. 1 is a diagram illustrating a communication system for the transmission of patient data to a clinician's station. The system shown is generic to transmissions from a clinician's office or from a remote location via a communication network of some kind. Medical data signals are acquired from a patient with an implantable medical device (IMD) 20 via telemetry head 22 or an equivalent device, and are uplinked to instrument 24. Communication between the IMD 20 and instrument 24 may take any wireless or broadcast format, including, for example, RF transmission over relatively long distances. Instrument 24 may represent a programmer in a clinician's office, or may be an in-home monitor adapter to communicate with IMD 20. Instrument 24 maintains wireless communication over link 27 to transfer data to clinician's station 28. Alternatively, instrument 24 may be adapted to transfer data via communication network 30, which may be Internet, extranet, worldwide web, or a similar type of network. The data is then transferred to clinician's station 28 by modem, cable, facsimile, or an equivalent data transfer system.

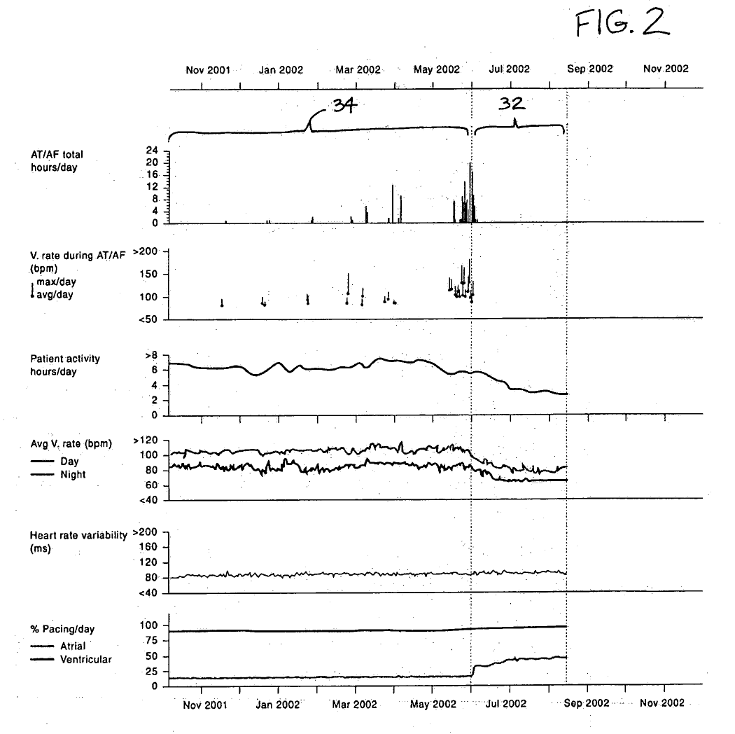

FIG. 2 is a Heart Failure Management Report, which is rendered over a 14 month time period. The Heart Failure Management Report includes time in atrial tachycardia (AT) or atrial fibrillation (AF), ventricular rate during AT/AF, patient activity, average ventricular rate, heart rate variability, and percent of pacing in the atrium and the ventricle. The report is assembled by combining data received in the current interrogation of the IMD (currently interrogated data 32) with historical data 34 stored in the memory of the IMD. Typical IMDs have a 14 month rolling memory, which dictate a 14 month report based on the information stored in that memory.

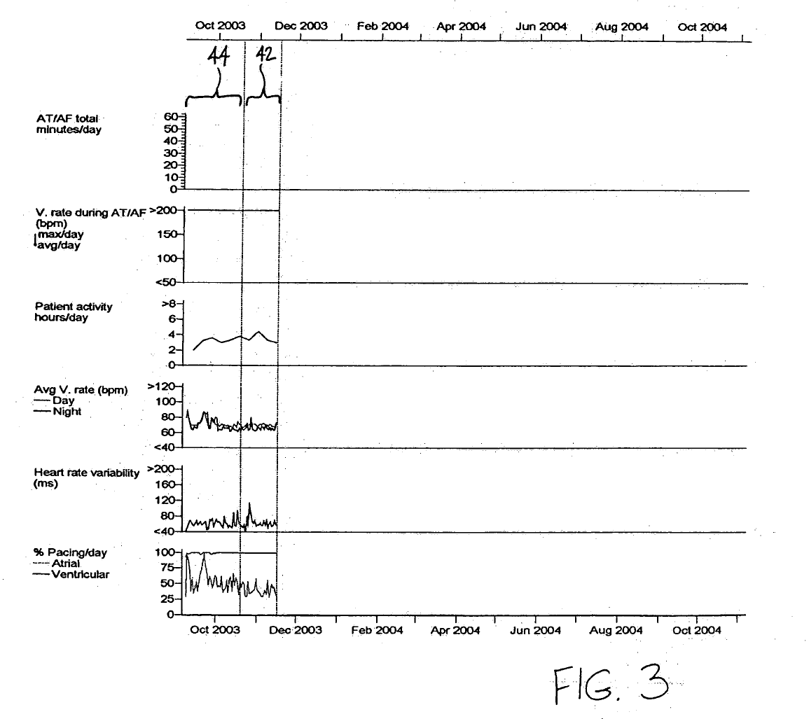

FIG. 3 is a Heart Failure Management Report that has 90 days of data, displayed on the 14 month time scale of a conventional report. This report is essentially the same as the report shown in FIG. 2, except that only 90 days of data (including currently interrogated data 42 and stored historical data 44) are available to be viewed. Currently interrogated data 44 makes up the last month of the data to be viewed, while historical data 42 makes up the remainder of the data shown. This scenario illustrates the difficulty of clearly viewing 90 days of data on the 14 month graph scale of the conventional Heart Failure Management Report, and particularly if the currently interrogated data (only the last month of data) is of special interest.

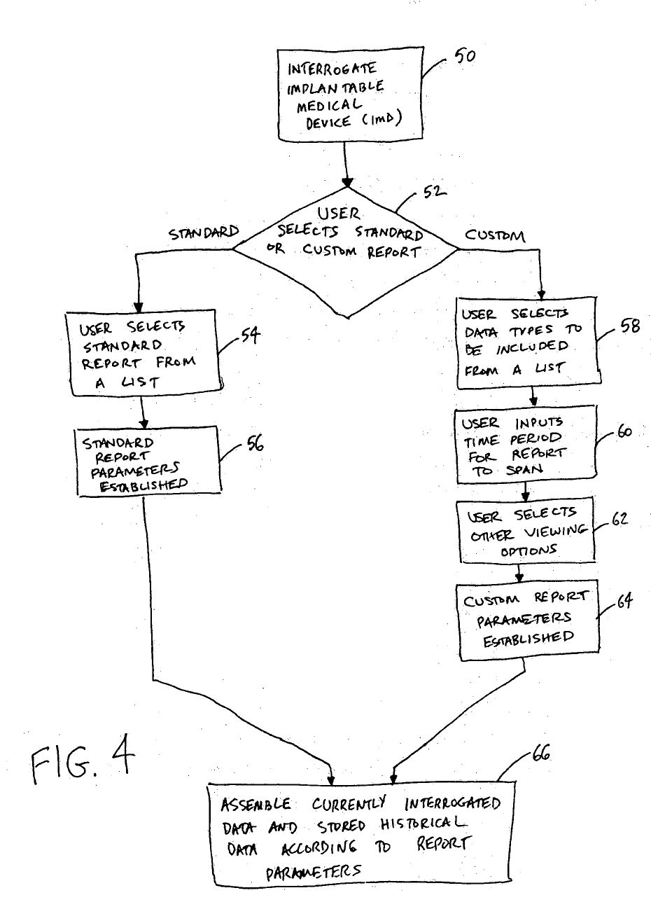

FIG. 4 is a flow diagram illustrating the process by which a user creates a custom report according to an embodiment of the present invention. Initially, an IMD is interrogated at block 50, to obtain currently interrogated data for inclusion in a report, along with stored historical data previously interrogated from the IMD. After interrogation, the user is able, at block 52, to select either a standard report (such as the report shown in FIG. 2) or a custom report. If the user selects a standard report, a particular standard report can be selected from a list, as indicated by block 54. Other ways to generate a standard report are known in the art; for example, the computer system may automatically generate one or more standard reports based on the data received. Once the standard report has been selected, the parameters associated with the standard report are established, as indicated at block 56.

If the user selects a custom report (block 52), there are several options for the user to specify for generation of the report. The user can specify the data types to be included in the report from a list of options, as indicated at block 58. The user can also input the desired time period for the report to span, as indicated at block 60. Other viewing options for the report, such as the scale for the x and y axes of particular graphs, superimposing options for independent data types or the same data type over different time period, calculations or statistical regressions based on the data displayed, and identification of trends or correlations between data types, may be specified at block 62. The parameters associated with the custom report are established at block 64 based on the selections and input provided by the user.

In an alternative embodiment, the user has the ability to automatically create a report that only displays data trends that have significant changes, with the threshold for such trends being defined by the user. The data types that are displayed only when significant changes occur can be integrated into a report that also displays other data types, or can form a stand-alone report that serves to alert the user when significant changes in data trends are detected.

Once the report parameters have been established, either for a standard report or for a custom report, the currently interrogated data and stored historical data from the IMD are assembled according to the report parameters to create the desired report, as indicated at block 66. By assembling the report after establishment of report parameters, rather than pre-rendering a static image representing a standard report, a great deal of flexibility is afforded to the user of the system, such as a clinician, in viewing the data received from the IMD. This flexibility allows the clinician to view any desired trends in the data, so that the clearest possible analysis of the patient's condition and need for follow-up care can be made. The custom report is available for display and for storage as an appropriate file.

In one embodiment, the custom report template created by the user (that is, the parameters and viewing options established by the user) can be saved as a file with an appropriate index or other identification, so that the custom report type can be used again without requiring the user to establish all of the parameters and viewing options every time the report is generated. The saved custom report file may be added to the list of standard reports, so that it can be selected by the user at block 52, or may be available to the user as part of the process of establishing parameters and viewing options at blocks 58, 60, 62 and 64.

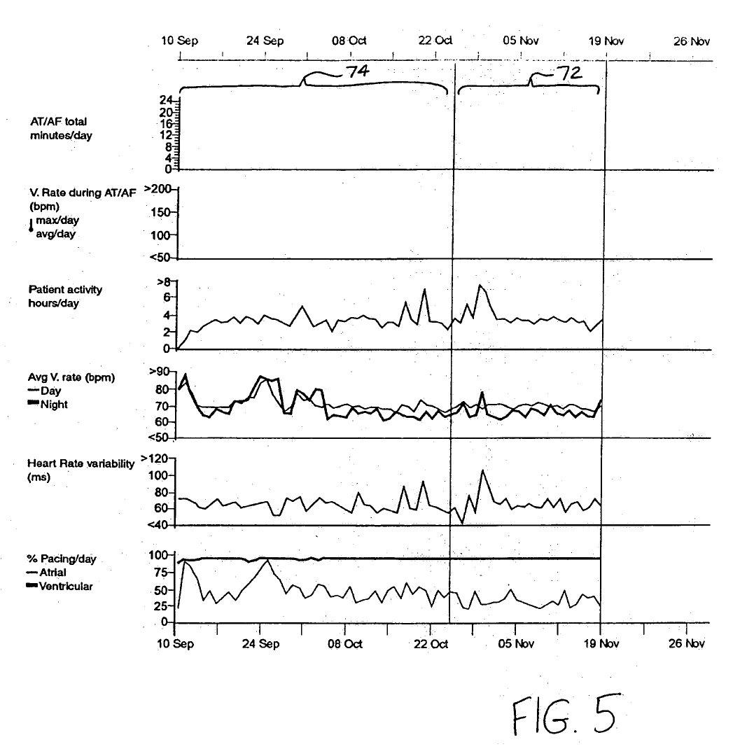

FIG. 5 is an exemplary custom Heart Failure Management Report created according to the process of FIG. 4. This custom report is created by a user to show data of interest over a time period of interest. In the example shown in FIG. 4, a report is created with a total scale of 90 days, and including time in atrial tachycardia (AT) or atrial fibrillation (AF), ventricular rate during AT/AF, patient activity, average ventricular rate, heart rate variability, and percent of pacing in the atrium and the ventricle. The data types shown in the report were selected by a user, and represent the standard data types for a Heart Failure Management Report. In one embodiment of the invention, a group of standard data types to be shown can be a particular option presented to the user for data type selection (block 58, FIG. 4), to make the process of creating the report as simple as possible. The time period of the custom report was input by the user as 90 days. The user's time input can be in units of days, weeks, months, years, or other increments of time. The 90 day report shown in FIG. 5 allows the user to clearly view short-term data trends, including the last month of currently interrogated data 72 (and historical data 74), so that appropriate analysis of the data can be performed.

In the management of patients with heart failure, it is desirable to prevent hospitalization as a result of fluid overload. This is a condition that can occur quickly in the matter of days or less. Clinicians need to be able to see daily changes in the trends of the Heart Failure Management Report in order to understand both historical and predictive data and correlate this data to a patient's symptoms. For example, a clinician has the ability to view a report of a symptomatic patient and change the patient's medications. Following the change in medication, the clinician is able to track patient activity, average ventricular rate and heart rate variability on a report that is customized to show this data on very short time scale (such as one day), to quickly determine the effectiveness of the newly prescribed medication. One such scenario involves the prescription of rate control medication for a patient with atrial arrhythmia. The rate control medication is intended to reduce the ventricular rate that is inappropriately high as a result of the atrial arrhythmia conducting from the atria to the ventricles. The clinician is able to create a custom, short-term report to perform a detailed examination of the ventricular rate during atrial tachycardia (AT) or atrial fibrillation (AF) to determine the effectiveness of the medication.

Although the present invention has been described with reference to preferred embodiments, workers skilled in the art will recognize that changes may be made in form and detail without departing from the spirit and scope of the invention.

1. A method of retrieving and presenting data from an implantable medical device (IMD), the method comprising:
interrogating the IMD to access the data;
establishing medical report parameters by user selection; and
assembling a medical report that includes only the data that corresponds to the selected medical report parameters.

2. The method of claim 1, wherein interrogating the IMD comprises accessing currently interrogated data and historical data stored in the IMD.

3. The method of claim 2, wherein the interrogated data and historical data stored in the IMD spans a time period of 14 months.

4. The method of claim 1, wherein establishing the medical report parameters comprises selecting at least one data type to be included in the medical report.

5. The method of claim 1, wherein establishing the medical report parameters comprises inputting a desired time period for the medical report.

6. The method of claim 5, wherein assembling the medical report comprises scaling the data corresponding to the desired time period for viewing.

7. The method of claim 1, wherein establishing the medical report parameters comprises selecting viewing options for the medical report.

8. The method of claim 1, wherein establishing the medical report parameters comprises selecting a standard report from a list of options.

9. A system for retrieving and presenting data from an implantable medical device (IMD), the system comprising:
a device for interrogating the IMD to access the data;
an instrument in communication with the device for transferring the data accessed by the device; and
a station for receiving the data from the instrument, the station being adapted to accept user selection of medical report parameters and assemble a medical report that includes only the data that corresponds to the selected medical report parameters.

10. The system of claim 9, wherein the data accessed by the device for interrogating the IMD includes currently interrogated data and historical data stored in the IMD.

11. The system of claim 9, wherein the interrogated data and historical data stored in the IMD spans a time period of 14 months.

12. The system of claim 9, wherein the instrument transfers the data to the station via a wireless communication link.

13. The system of claim 9, wherein the instrument transfers the data to the station via a communication network.

14. The system of claim 9, wherein the device and the instrument comprise an in-home monitoring system and the station is in a remote location.

15. The system of claim 9, wherein the medical report parameters comprise at least one data type to be included in the medical report.

16. The system of claim 9, wherein the medical report parameters comprise a desired time period for the medical report.

17. The system of claim 16, wherein the medical report is scaled according to the desired time period for viewing.

18. The system of claim 9, wherein the medical report parameters comprise user-defined viewing options.

19. The system of claim 9, wherein the medical report parameters are defined by user selection of a standard report from a list of options.

20. The system of claim 9, wherein the medical report parameters are defined by user selection of a previously created custom report.
